(19) United States
(12) Patent Application Publication
(10) Pub. No.: US 2014/0368819 A1
(43) Pub. Date: Dec. 18, 2014

(54) Methods for Detecting Parasites, Viruses, Bacteria and Drugs in Human and Animal Blood and Cerebral Spinal Fluid, Using Laser-Induced Breakdown Spectroscopy

(71) Applicant: Applied Research Associates, Inc., Albuquerque, NM (US)

(72) Inventors: Rosalie A. Multari, Albuquerque, NM (US); David A. Cremers, Santa Fe, NM (US)

(21) Appl. No.: 14/303,313

(22) Filed: Jun. 12, 2014

Related U.S. Application Data
(60) Provisional application No. 61/834,002, filed on Jun. 12, 2013.

(51) Int. Cl. G01N 33/49; G01N 21/25; G01N 21/01; A61B 5/00

(52) U.S. Cl. CPC G01N 33/49; A61B 5/4845; G01N 21/255; G01N 21/01; G01N 2021/0162
USPC 356/318

(57) ABSTRACT

The present invention relates to methods of detecting parasites, viruses, bacteria and drugs in human and animal blood and cerebral spinal fluid (CSF), using laser-induced breakdown spectroscopy (LIBS). The method includes developing and using algorithmic detection models for detecting compounds, bacteria, viruses and parasites in blood or CSF. The models are developed from a sample of blood or fluid, knowingly having one or more of the compounds or bacteria, viruses, or parasites. Spectra are generated by a LIBS instrument from the sample, and are grouped into either classification spectra or verification spectra. Algorithmic models are developed from the classification spectra; these models are verified with the verification spectra. A second sample of different blood or CSF may then be assessed using the algorithmic models. Spectra generated from this second sample are applied to the models to determine the presence or absence of compounds or bacteria, viruses and parasites of interest.

### CROSS REFERENCE TO RELATED APPLICATIONS

The present application claims priority to and the benefit of U.S. Provisional Patent Application No. 61/834,002, filed Jun. 12, 2014, the disclosure of which is incorporated herein by this reference.

### BACKGROUND OF INVENTION

The present invention relates to a method of detecting parasites, viruses, bacteria and drugs in human and animal blood and cerebral spinal fluid (CSF), using laser-induced breakdown spectroscopy (LIBS).

LIBS is a form of atomic emission spectroscopy (AES) used to perform elemental analysis. Instrumentation that performs LIBS include a laser, a receiver and a spectrometer. To perform this analysis using such a device, a laser pulse is directed to a sample to be analyzed, generating a plasma on the surface of the sample, and atomizing (vaporizing) and exciting the sample. When atoms and ions of an element are atomized and excited, they emit a unique ‘fingerprint’ spectrum or characteristic wavelength useful for identification. Thus, elements can be identified in a sample by collecting the light emitted from the plasma by the LIBS device receiver and spectrally analyzing the received light by means of the spectrometer, to extract information needed to determine the composition or identity of the sample.

To date, LIBS has not been used to determine the composition or identity of human or animal blood or CSF, and specifically to determine the presence or absence of parasites, viruses, bacteria or drugs. Therefore, there is a need in the art to provide rapid, in-situ clinical diagnostic assessment and identification of compounds and pathogens within blood and CSF by means of laser-induced breakdown spectroscopy.

### SUMMARY OF THE INVENTION

By means of the present invention, LIBS provides rapid, in-situ clinical diagnostic assessment and identification of compounds and pathogens within blood and CSF.

To provide such a detection system, the present invention first develops algorithms from LIBS spectra collected directly from blood or CSF infected with parasites, viruses, bacteria or drugs. The spectra are analyzed using elemental, molecular and background emissions over a wide spectral range (e.g., 200-1000 nm) or selected wavelength ranges, and from this analysis detection algorithms are developed for deployment on LIBS instrumentation. Thereby, using such algorithms the LIBS device can detect parasites, viruses, bacteria or drugs in blood or CSF, allowing for rapid, in-situ clinical diagnostic assessment and identification of compounds, parasites, bacteria and viruses within blood and CSF.

The detection algorithms may be developed using methodology described in U.S. Pat. No. 8,655,807, issued to Applied Research Associates, Inc., on Feb. 18, 2014, the disclosure of which is incorporated by this reference. By such methods, algorithms can be developed for compound and pathogen identification within a blood or CSF sample, using multivariate analysis applied to the entire LIBS spectrum or a subset wavelength range(s) of the spectrum. These algorithms provide ‘spectral fingerprints’ to identify biological targets of interest for a specific equipment sampling configuration; the detection algorithms developed are instrument, application and sample specific.

The present invention is generally a method for detecting parasites, viruses, bacteria or drugs in human blood, animal blood or CSF. The method uses and assesses the presence of these foreign substances in a sample of human blood, animal blood or CSF. Such assessment includes, by means of a LIBS instrument, applying a laser to the sample, collecting light emitted from said sample, and generating spectrum from said collected light. Once the spectra are generated, the method and device of the present invention determines the presence or absence of parasites, viruses, bacteria or drugs, or combinations thereof, in the sample, by applying one or more algorithmic models to the generated spectra. The algorithmic models are developed from other spectra generated by a LIBS instrument from samples of human blood, animal blood or CSF with known presence of parasites, viruses, bacteria or drugs.

A LIBS device useful in the present invention includes a computer having algorithmic detection models for detecting parasites, viruses, bacteria or drugs in human blood, animal blood or CSF. These algorithmic models are developed from and verified by spectra generated by a LIBS instrument from samples of human blood, animal blood or CSF with known presence of parasites, viruses, bacteria or drugs.

Also provided is a method for developing and using algorithmic detection models for detecting parasites, viruses, bacteria or drugs in human blood, animal blood or CSF. In this embodiment, the method for developing the algorithmic detection models begins with obtaining a first sample of human blood, animal blood or CSF, knowingly having one or more parasites, viruses, bacteria or drugs. Then, using a LIBS instrument, a laser is applied to the sample, light emitted from the sample is collected, and spectra from the collected light are generated. These generated spectra are then grouped into either classification spectra or verification spectra. From the classification spectra, one or more algorithmic models are developed; these models are verified with the verification spectra. The use of the developed algorithmic detection models also begins with providing a second sample of different human blood, animal blood or cerebral spinal fluid. The generated spectra are then applied to the algorithmic models to determine the presence or absence of parasites, viruses, bacteria or drugs, or combinations thereof, in the second sample.

As disclosed in the '807 Patent, spectral data from a sample or group of samples is collected and divided into two sets—a classification set and a verification set. Predictive models are then created with the classification set, on a group by group comparison using mathematical analysis. The performance of the models are tested using the verification set. By combining models constructed in this way in a logical flow, detection algorithms can be generated and deployed on a LIBS instrument to determine the identity of an unknown sample (see the '807 Patent for details).

By use of these methodologies to detect parasites, viruses, bacteria and drugs in human and animal blood and cerebral spinal fluid, your inventors have found that LIBS can be used for rapid, in-situ assessment and identification of compounds and pathogens within blood and CSF.

### DESCRIPTION OF DRAWINGS

FIGS. 1A and 1B are schematics of LIBS instruments suitable for use in the present invention.

FIG. 2 shows prediction value results for verification spectra input into a mathematical differentiation model created to differentiate LIBS spectra collected from a blood sample containing a parasite (Leishmania donovani (LD)) from blood samples containing a bacteria (S. aureus), a virus (HIV), parasite media, virus media, and uncontaminated whole blood, wherein the top plot shows differentiation of all but two control media samples (parasite media), and the bottom plot shows prediction value results from a second model created to differentiate the parasite from the parasite media. By combining these two models in a logical flow, it is possible to identify the blood samples containing the parasite.

FIG. 3 shows prediction value results for verification spectra input into a single mathematical differentiation model created to differentiate LIBS spectra collected from a human blood sample containing a bacterium from samples of uncontaminated human blood and human blood containing a parasite.

FIG. 4 is an illustration of the structure of an algorithm designed to differentiate a bacterium, a parasite, and a virus in human blood.

FIG. 5 shows prediction results for verification spectra input into a model designed to differentiate a first vaccine (M) from a second vaccine (A) and a mixture of the first and second vaccines (AM) in pig's blood.

FIG. 6 is an illustration of the structure of an algorithm designed to differentiate a virus in CSF.

FIGS. 7A and 7B show prediction results for verification spectra when input into models for differentiating a blank filter and a filter with bacteria media.

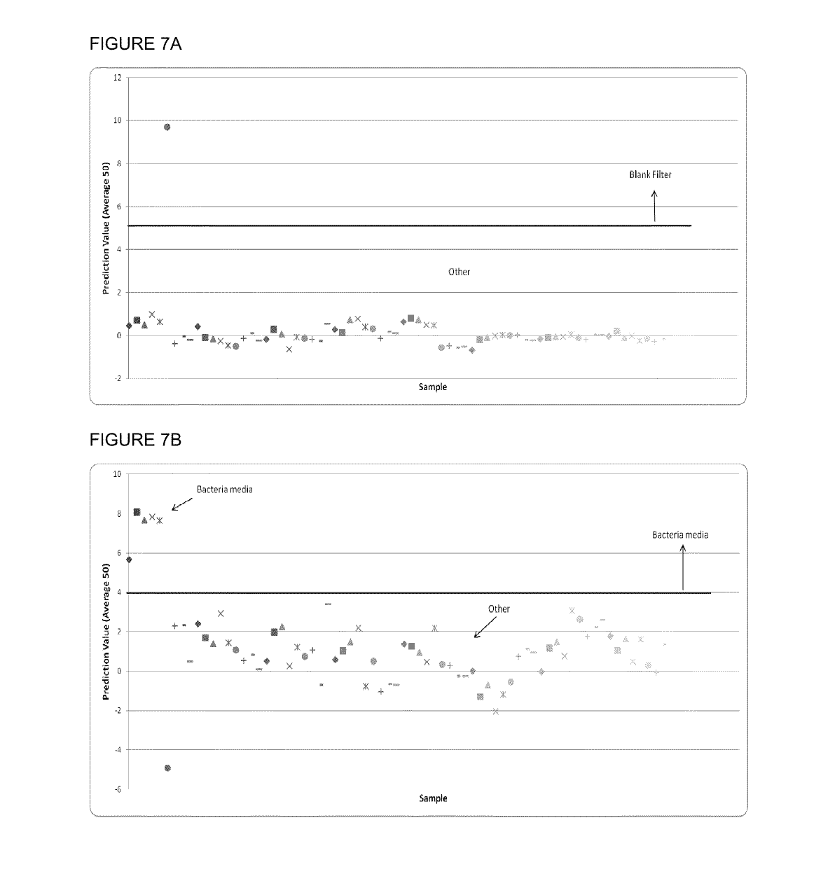

FIGS. 8A, 8B and 8C show prediction results for verification spectra when input into models designed to differentiate parasite concentration in human blood.

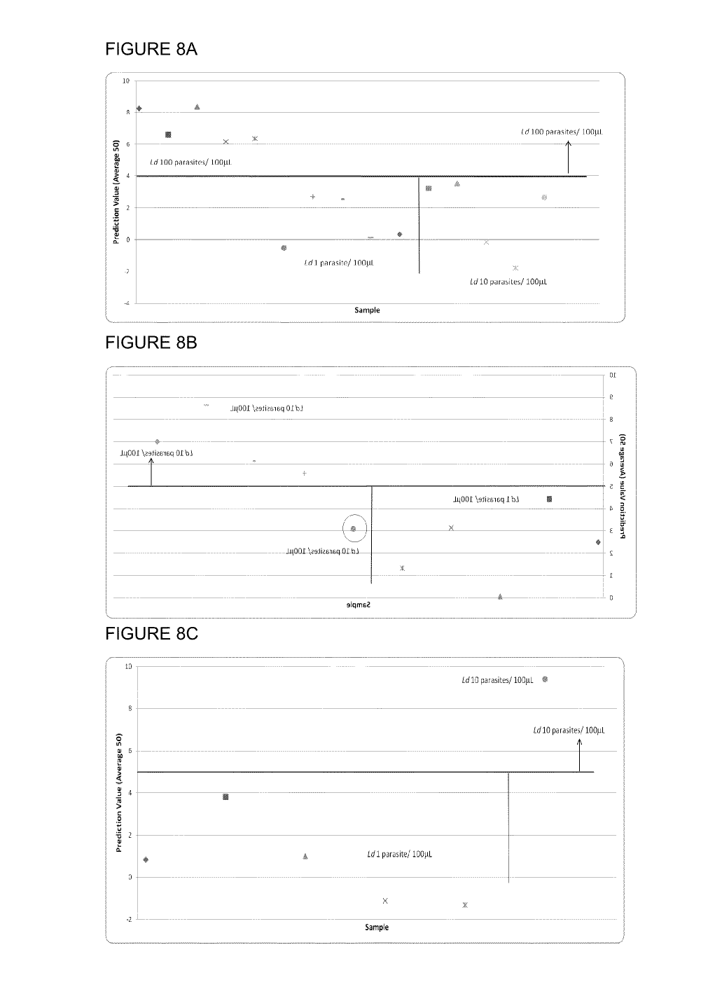

FIGS. 9A and 9B show prediction results for verification spectra when input into models designed to differentiate blood with parasite from uncontaminated blood, blood with virus, blood with virus media, and blood with bacteria; and HIV at various concentrations and virus media together uncontaminated blood, blood with different concentration of virus, and uncontaminated blood.

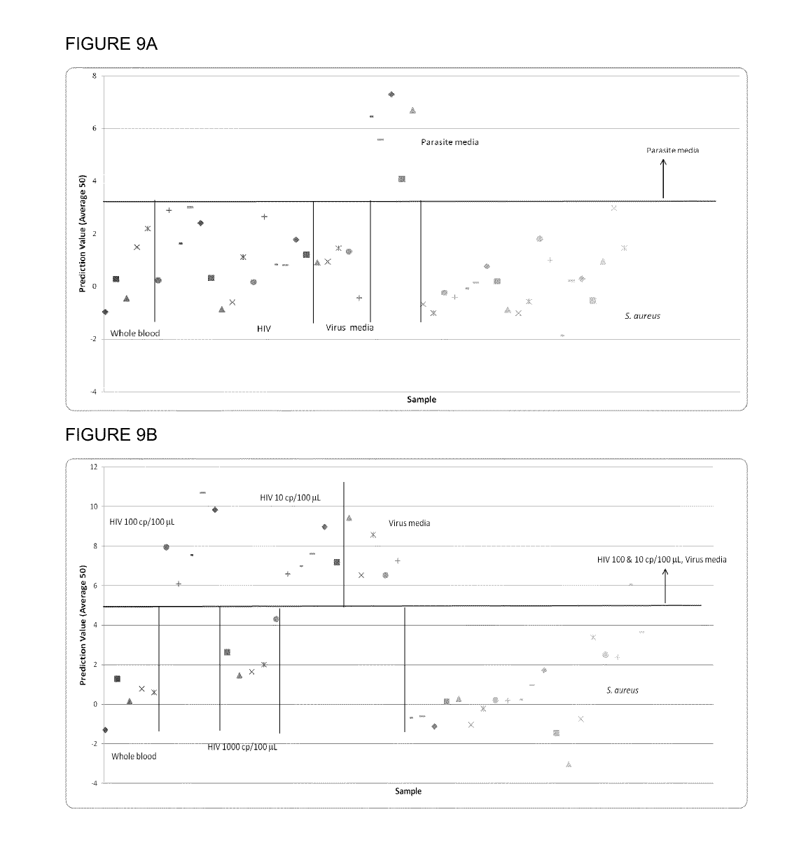

FIGS. 10A and 10B show prediction results for verification spectra when input into models for differentiating virus media from the group, and an HIV concentration.

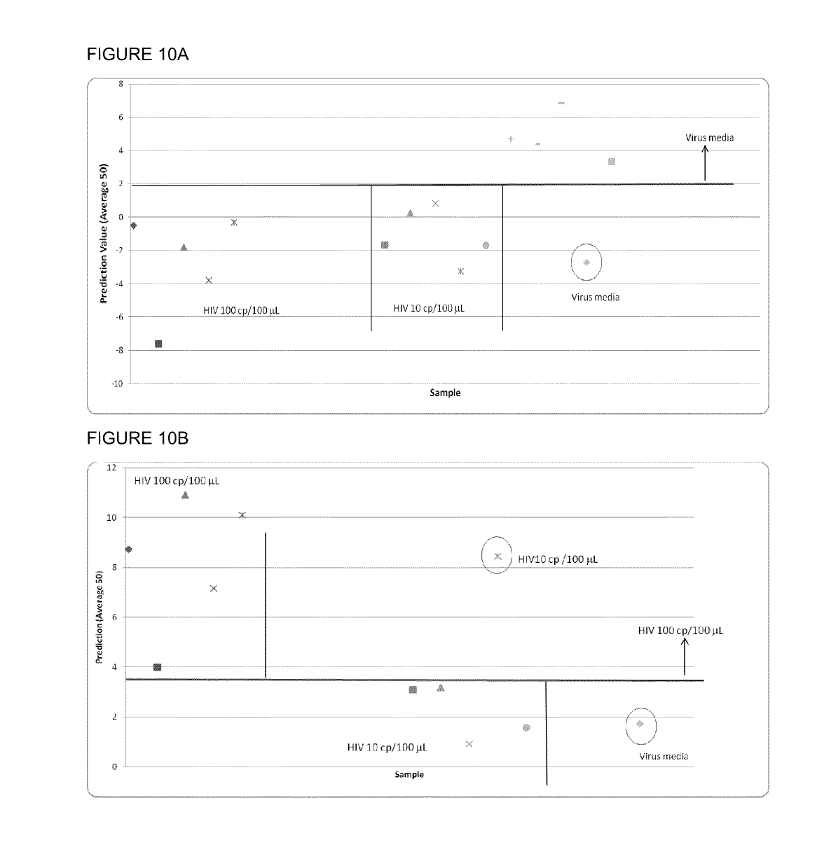

FIGS. 11A and 11B show prediction results for verification spectra from two models designed to differentiate the highest concentration of HIV when used together.

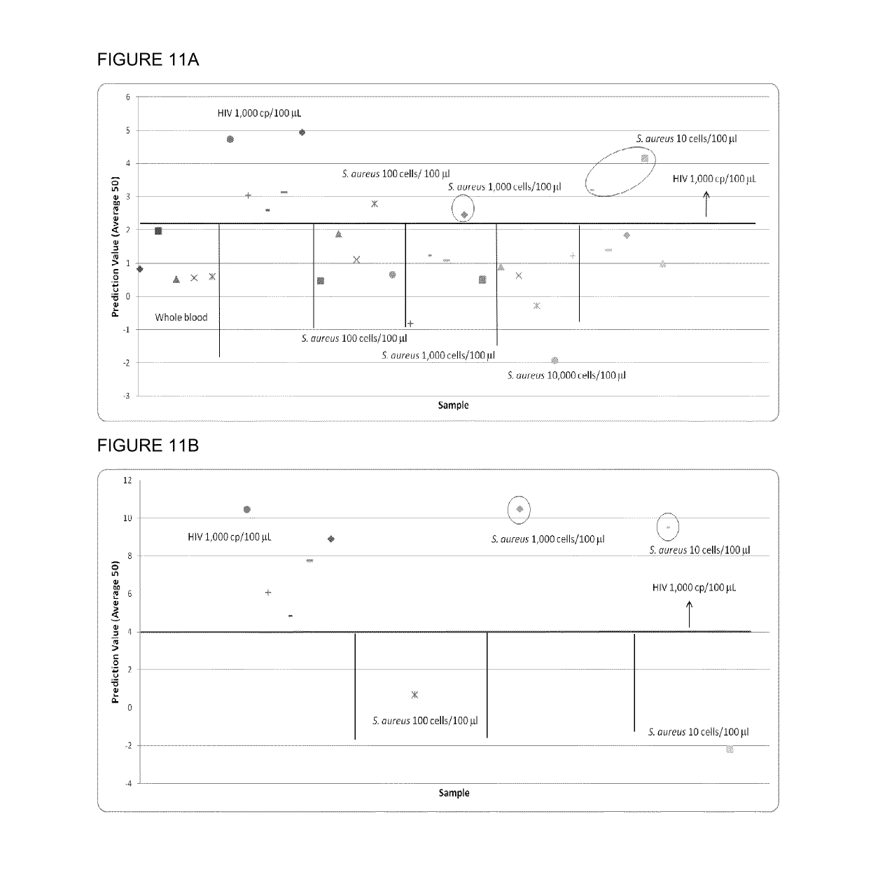

FIGS. 12A and 12B show prediction results for verification spectra from two models designed to differentiate bacteria when used together.

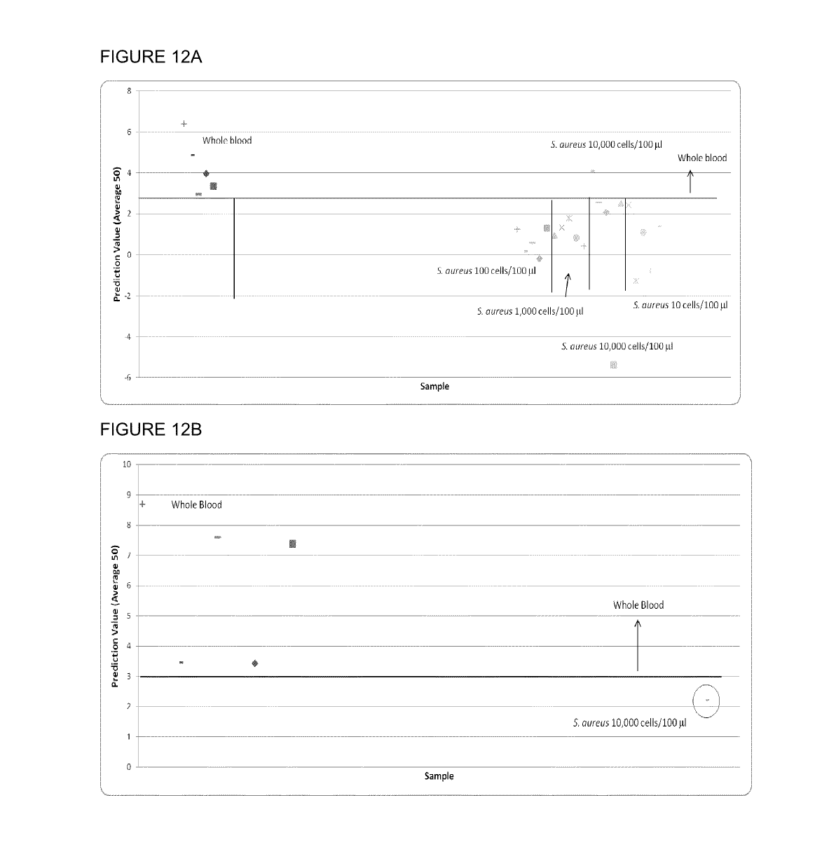

FIGS. 13A, 13B and 13C show prediction results for verification spectra when input into models for differentiating between bacteria concentrations in blood.

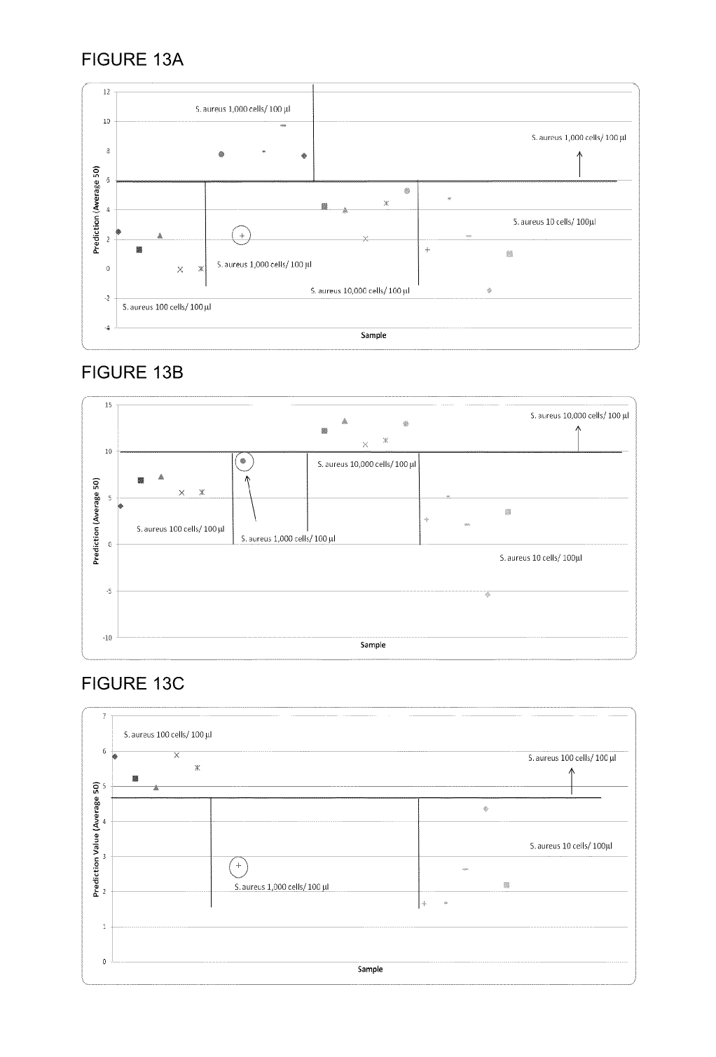

FIGS. 14A and 14B show prediction results for verification spectra when input into models for differentiating a bare slide with nothing on it and a slide with cell culture media in the algorithm for detecting herpes simplex in CSF.

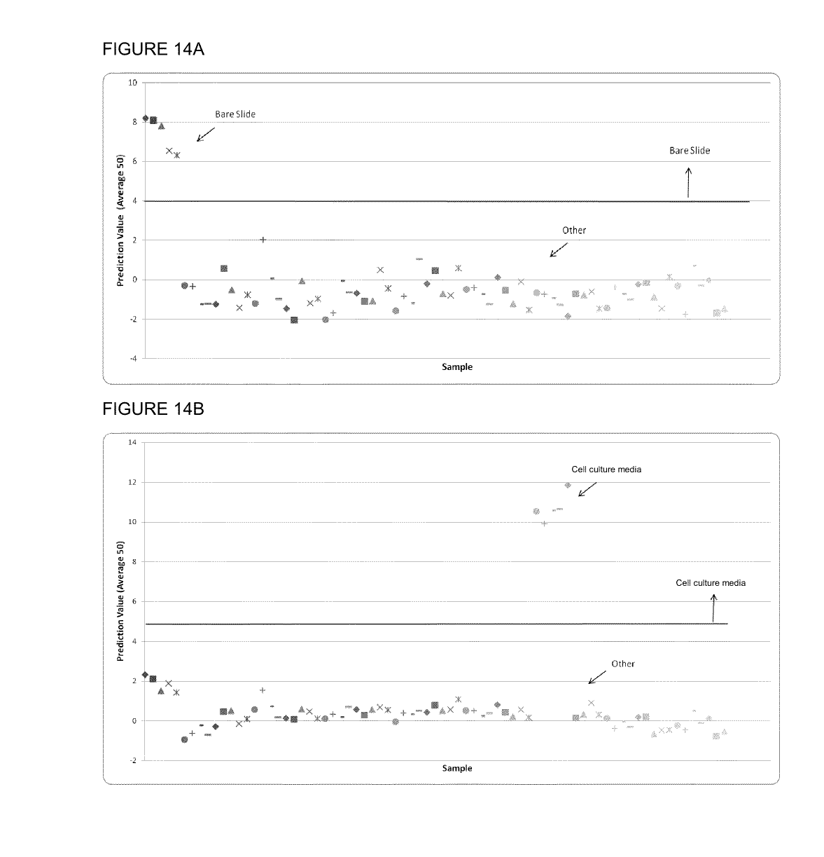

FIGS. 15A and 15B show prediction results for verification spectra when input into models for differentiating CSF and HSV or cell culture media dilution.

FIGS. 16A and 16B show prediction results for verification spectra when input into models for differentiating first HSV1 from HSV2 and cell culture media, and second HSV2 from cell culture media.

FIGS. 17A, 17B and 17C show prediction results for verification spectra when input into models for differentiating HSV1 by concentration from cell culture media when used together.

### DETAILED DESCRIPTION

The present invention utilizes the methods of the '807 Patent to develop algorithms to detect parasites, bacteria, viruses, and drugs in blood or CSF; the detection algorithms are then deployed on LIBS instrumentation.

The detection algorithms are developed using calibration data collected from the various samples in the detection groups that the LIBS instrument is designed to differentiate (e.g. S. aureus, Leishmania donovani (Ld), and Human immunodeficiency virus (HIV) in blood and uncontaminated blood; or Herpes simplex virus (HSV) in CSF and uncontaminated CSF). Once developed, the algorithms are application specific. This means the algorithms can only be used for detection of the parasites, bacteria, viruses or drugs in either blood or CSF on the substrate on which they are deposited (e.g. filter paper or glass) and for the sample condition (e.g. wet or dry) for which the data were collected to develop the algorithm. However, multiple detection algorithms can be deployed on a single instrument through programming with the choice of which detection algorithm(s) to use determined by the operator or programmed into the instrument. Thereby, the operator may select the detection algorithms stored on the LIBS apparatus based upon the sample (blood or CSF), its substrate and its condition, as well as the parasites, bacteria, viruses or drugs to be detected if present.

Because the shape of the spectral data collected and from which the algorithms are derived is specific to the instrument configuration, the algorithms can be used only for the instrument configuration from which the calibration data were collected. Spectra shape is specific to not only the configuration of the instrument, for example the lens to sample distance and the relevant equipment parameters of the laser, such as the laser energy, the geometry and characteristics of the optical system used to focus the laser pulse onto the samples and to collect the plasma light, the type of spectrometer, etc., but is also a function of the condition of the sample (e.g., wet, dry, etc.), and the substrate material to which the sample is applied (e.g. filter paper or glass). Changing any of these parameters changes the shape of the recorded LIBS spectrum and therefore impacts the performance of the detection algorithm deployed on the LIBS instrument (e.g. algorithm for the detection of HIV in blood on filter paper).

The LIBS detection algorithm for detection of parasites, viruses, bacteria or drugs in blood or CSF can be constructed of one or more mathematical models to be used alone or in combination with each other and/or with traditional elemental analysis. A detailed description of the method used for multi-model detection algorithms may be found in the '807 Patent.

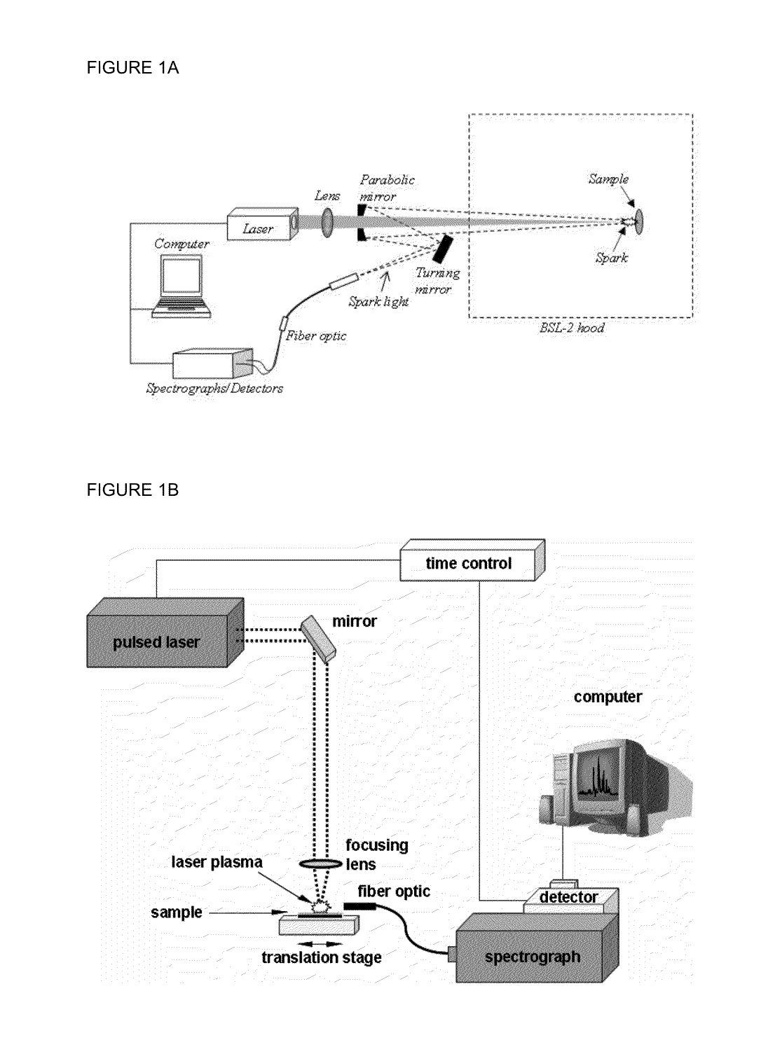

The basic development procedure for the detection algorithm is independent of the method chosen for analysis. Analysis methods for building the detection models include but are not limited to methods such as partial least square regression, neural networks, etc. The development procedure comprises collecting LIBS spectra from samples of blood or CSF with and without the parasites, bacteria, viruses, or drugs of interest at one or more concentrations (other types of samples of interest can also be included in the differentiation if there is a desire to specifically screen for them). One possible configuration of a LIBS instrument for data collection is shown in FIGS. 1A and 1B.

A sufficient number of individual spectra (e.g. 100 spectra, each the average of individual spectra (e.g. 10) for each sample used to develop the algorithm) are collected from each sample to capture the variability in spectral data characteristic of the samples and instrument in use. Typically, more than one sample of each type (e.g. parasite, virus, etc.) will be used to collect spectra (e.g. 5 replicate samples of each type) so that some sample variability can be built into the analysis.

The spectra collected for each sample are then divided into two groups: the first group for developing mathematical differential model(s) to be used in the detection algorithm and the second group for test and verification of the model(s) performance. Spectral normalization (such as normalization to the highest intensity feature in the spectrum or normalizing to total area, etc.) may be applied as needed to compensate for fluctuations in the spectral intensities due to both sample and equipment variability.

Mathematical analysis techniques such as chemometric analysis, multivariate regression analysis, neural networks, etc., and/or combinations thereof are applied to the collected spectra to build a differential model capable of discriminating between the samples. Using a single model or a set of models, an analysis algorithm for detection is created using the model(s) in a defined flow and/or in combination with elemental or other types of spectral analysis.

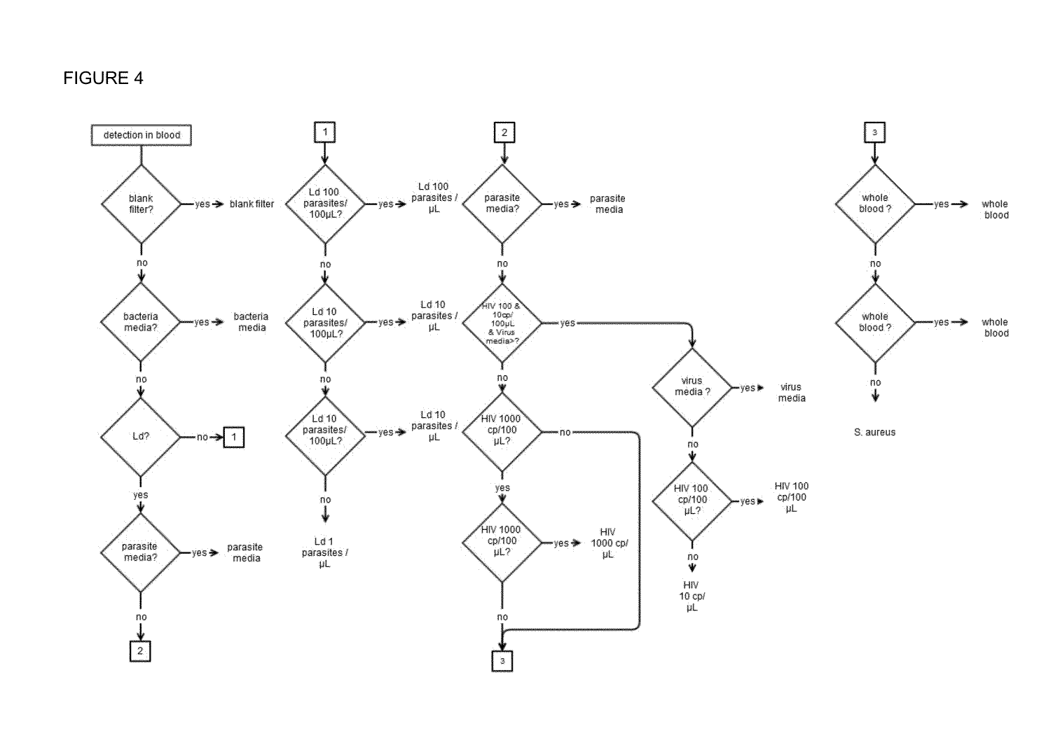

An embodiment of a combination of models used to detect parasites, bacteria and HIV in blood is shown in FIG. 4. In this analysis, models are sequentially run to determine, based upon spectral analysis, whether the sample is a blank filter, and if not whether it is a bacteria media. If neither, then the algorithm then determines whether Ld is present, and if so determines whether a parasite media is present. If Ld is present, the concentration of Ld is determined by means of multiple models. If the no Ld is present, and the specimen is not a parasite media, then the analysis continues by discriminating HIV by concentration, testing for virus media, and determining whether whole blood is present by means of two different models to screen for high concentrations of HIV.

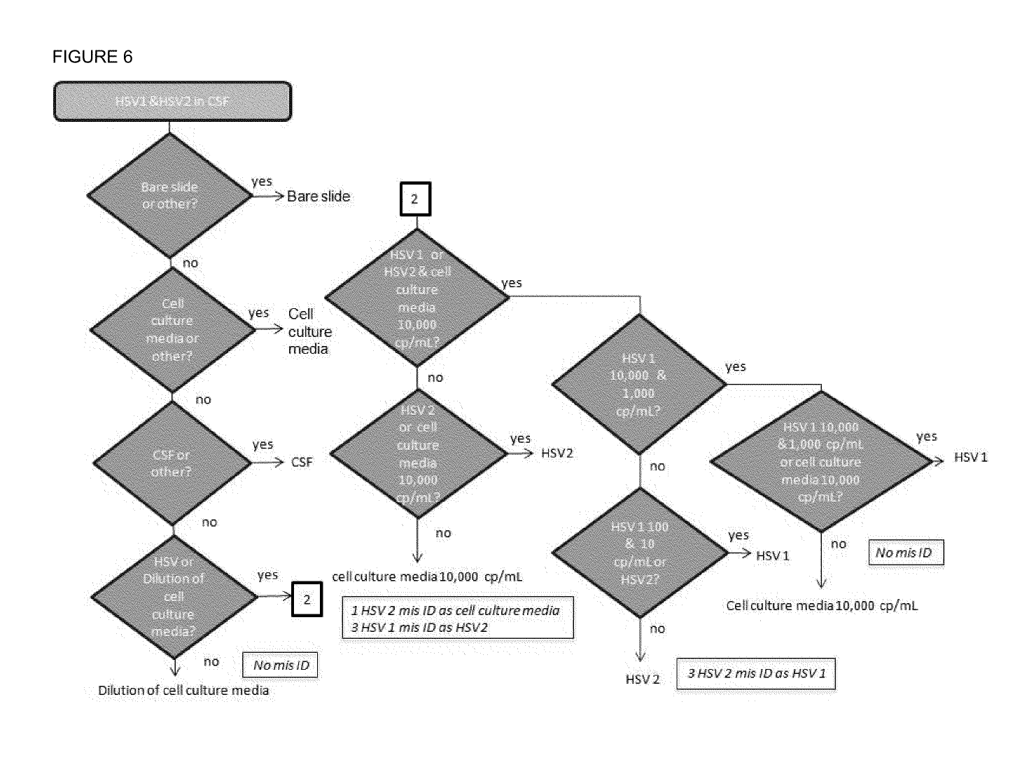

Another embodiment of a combination of models used to differentiate HSV in cerebral spinal fluid is shown in FIG. 6. Each block represents a different model in the flow and multiple models are needed for screening some of the sample groups. In this series of models, by means of spectral analysis the present invention determines whether the sample is a bare slide; if not, whether it is a cell culture media, and if not whether it is CSF. Once CSF is determined, HSV and dilution of cell culture media are discriminated, HSV 1 is discriminated from HSV2 and cell culture media, and HSV2 is discriminated from cell culture media. The concentration of HSV 1 is then determined. This algorithm demonstrated 98% HSV differentiation with 100% specificity (all HSV samples were identified as HSV, but some non HIV samples were misidentified as HSV).

The models are tested on the set of spectra reserved to evaluate its performance and refined as needed until desired identification performance is achieved (e.g. >95% correct identifications). Statistical processing such as averaging the prediction values prior to sample differentiation may also be used to improve detection performance. The number of prediction values that are averaged will depend on the observed measurement-to-measurement fluctuations when the verification spectra are input into the differentiation models.

Sample identification may require both model prediction values (or other modeling results such as standard deviation) to be within a certain range and a particular elemental peak to be observed in the spectra of the sample as opposed to only relying on the model prediction values as discussed in the '807 Patent. This analysis algorithm is subsequently deployed on the LIBS instrument.

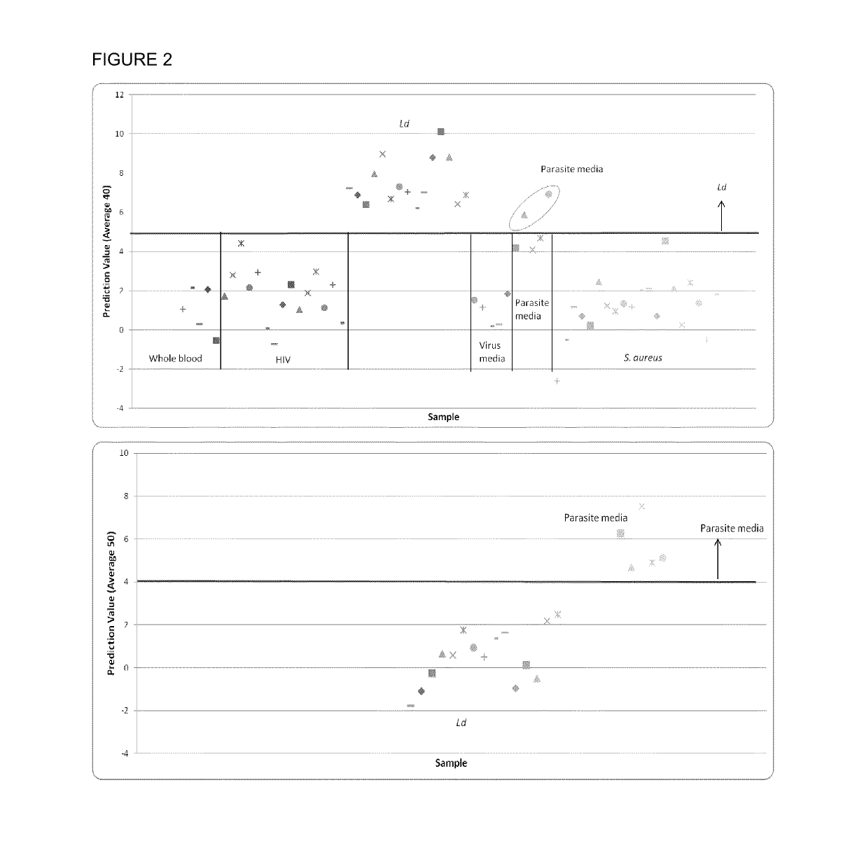

An example of verification spectra results from models created to differentiate a parasite Leishmania donovani in human blood from uncontaminated human blood, human blood containing a bacterium media (control), containing the bacterium S. aureus, containing a virus media (control), containing HIV, and containing a parasite media (control) may be found in FIG. 2. All but two control media samples (parasite media) are differentiated in the model of the top plot of FIG. 2; the bottom plot shows prediction value results for the same verification spectra when input into another model created to differentiate the parasite from the parasite media. These verification spectra were not included in the model development and the model was developed using only ⅗ of the samples of each sample type included in the verification set so detection is demonstrated on samples for which no corresponding spectra were included in the modeling. By using both models sequentially in a detection algorithm, the blood sample containing the parasite can be differentiated from uncontaminated blood and blood containing the bacterium and the virus. In some cases, detection can also be achieved using a single model instead of multiple models.

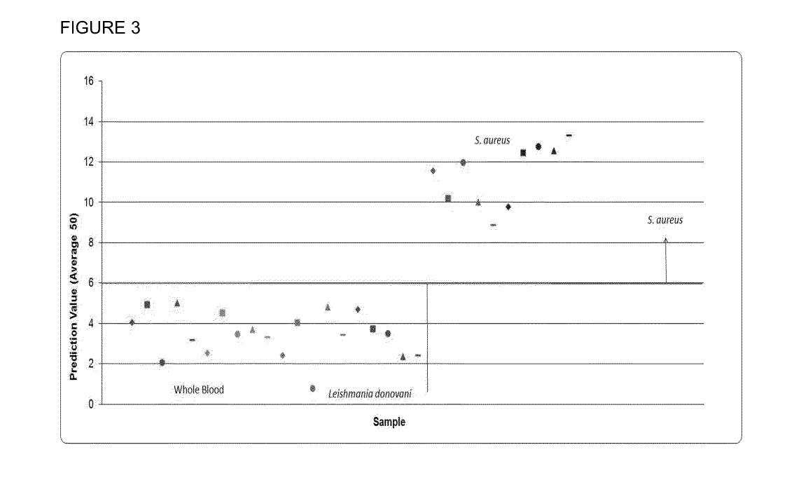

FIG. 3 shows prediction results for verification spectra input into a single mathematical differentiation model created to differentiate LIBS spectra collected from a human blood sample containing the bacterium S. aureus, from uncontaminated blood and blood containing the parasite Leishmania donovani. As in FIG. 2, the spectra used to test the models were not included in the model development and the model was developed using only ⅗ of the samples available for model build but tested over all of the samples. Similar models were constructed to differentiate the parasite from the uncontaminated blood samples.

FIG. 4 contains an illustration of the structure of an algorithm designed to differentiate a bacterium, a parasite, and a virus in human blood. Each block represents a different model in the flow and multiple models needed for screening some of the sample groups. This algorithm demonstrated 100% Leishmania donovani specimen discrimination, 100% uncontaminated blood differentiation, 100% HIV sample differentiation with a specificity of 95% for HIV (some non-HIV samples were misidentified as HIV), and 90% S. aureus differentiation.

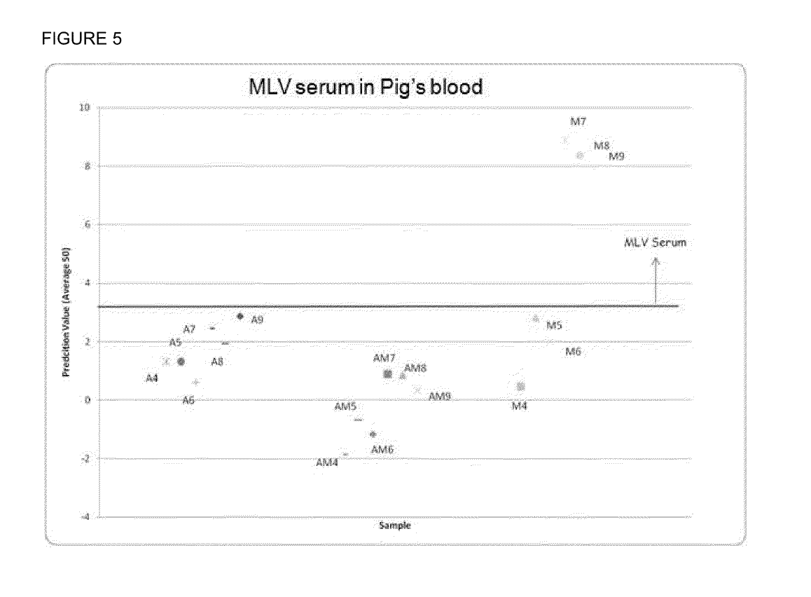

FIG. 5 demonstrates a model to be used in a discrimination algorithm for the detection and differentiation of a drug in pig's blood. In this example, the presence of a vaccine (MLV vaccine, M) is detected and differentiated from another vaccine (ATP vaccine, A) and a mix of the two vaccines (AM). These verification spectra, as in the other examples, were not included in the model development. As not all of the drug samples are screened by this model, additional models would follow to completely screen for the drug.

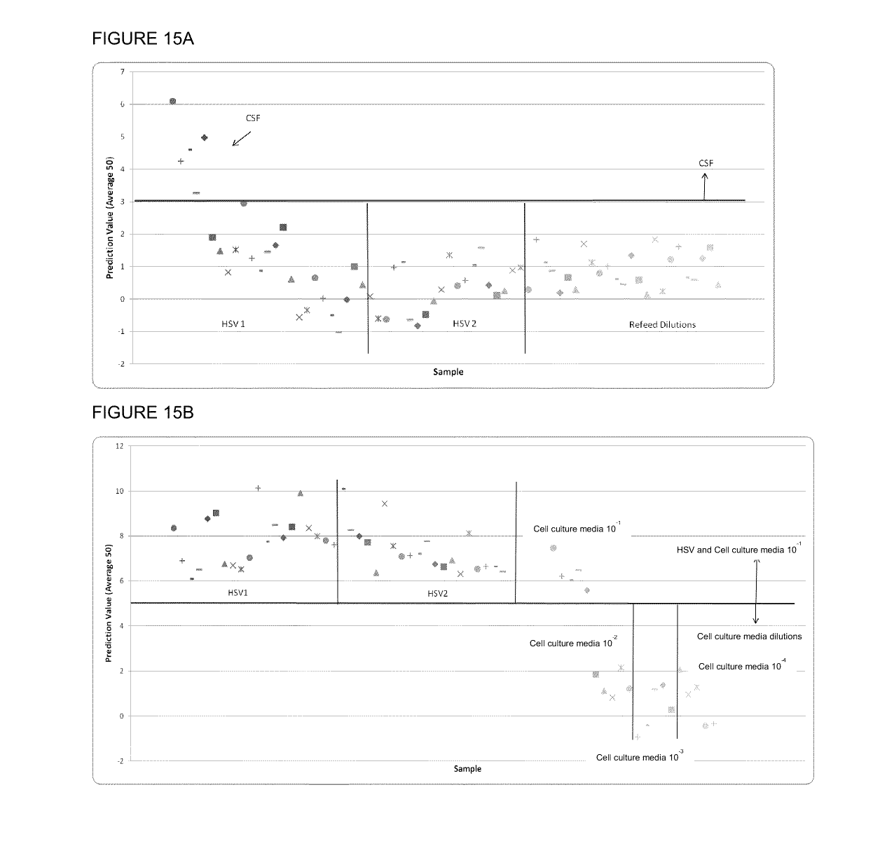

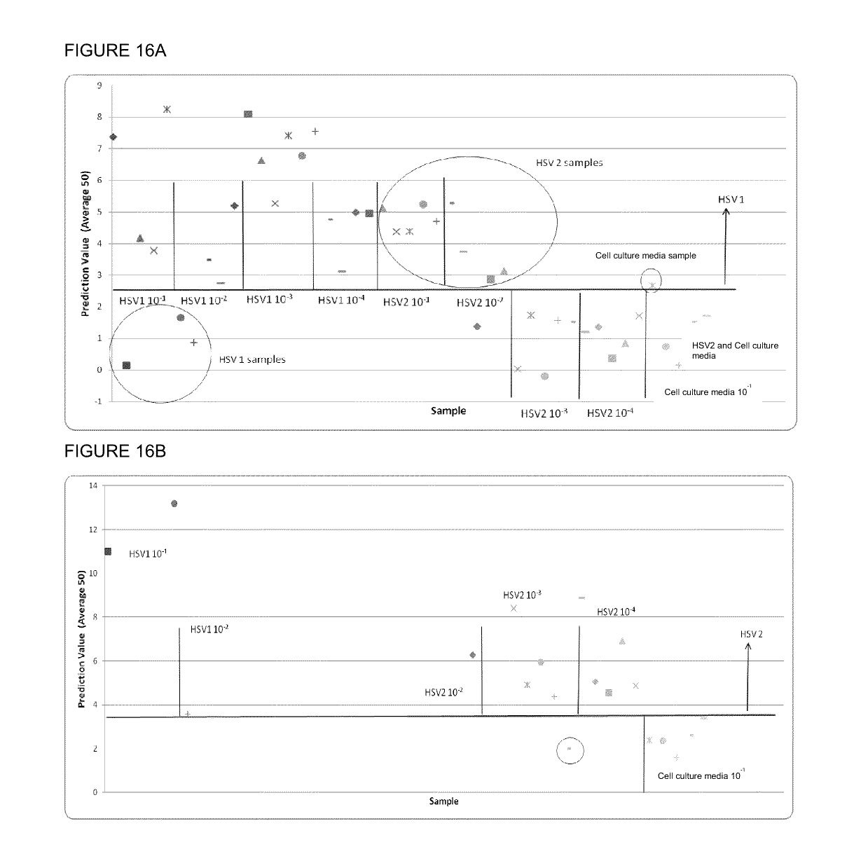

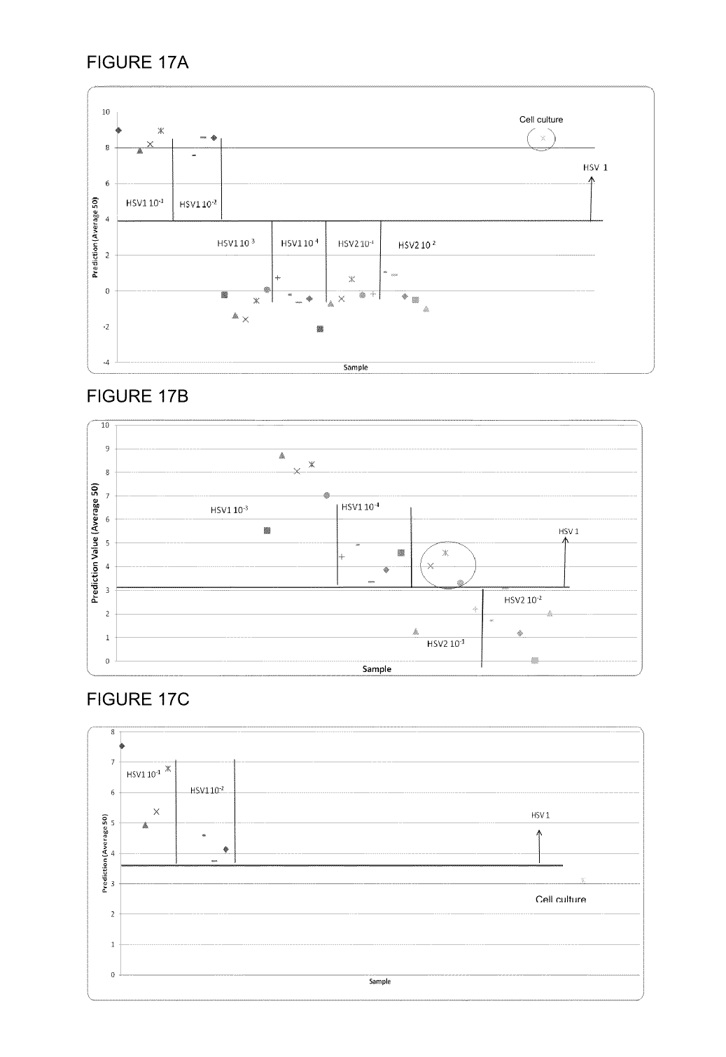

FIGS. 15-18 contains the results of inputting LIBS verification spectra into four differentiation models that were created to be used in combination with each other to differentiate HSV in cerebral spinal fluid from uncontaminated cerebral spinal fluid and a cell culture media control. In this example, first the pure CSF samples are differentiated from the other samples, then all but one of the CSF dilution samples are differentiated from all of the HSV samples, then all the HSV1 dilution and two of the HSV2 dilution samples are separated from the culture media samples and the two highest dilution HSV2 samples, and lastly, the remaining HSV2 dilution samples are separated from the remaining CSF dilution samples. Thus, differentiation of all of the samples containing HIV from samples not containing HIV is demonstrated. Additional models in the flow that are not shown can be used to discriminate for the type of HSV.

### EXAMPLE 1

### Staphylococcus aureus, Leishmania donovani (Ld) and Human Immunodeficiency Virus in Whole Blood

In this example, five replicate samples were prepared for each sample group, diluted in blood and applied to filter paper. The Ld dilutions in blood were between 1-10² parasites/100 μL, to achieve differentiation of specimens diluted to contain varying levels of parasite concentrations; the S. aureus dilutions in blood were between 10-10⁵ cells/100 μL; the HIV dilutions in blood were between 10-10³ cp (copies)/100 μL. Modeling over three samples, performance check over all five samples. After testing for bacteria media, the system tested for Ld via two models, to differentia Ld from the bacteria, virus and media samples. Once the Ld samples have been differentiated as a group, the Ld concentration can be differentiated (e.g., 100, 10 or 1 parasites/100 μl). This testing yielded performance of Ld in whole blood algorithm 100% correct in the overall identification of varying levels of parasite concentrations including Ld media, LD 1 parasite/100 μl, Ld 10 parasites/100 μl and Ld 100/100 μl.

The system also included models for detecting parasite media, the presence of HIV at concentrations of 100 and 10 cp/100 μl is present, and if so whether a virus media is present. If the concentration levels aren't found in the sample, the system of this embodiment proceeds to determine whether HIV is present at 100 cp/100 μl, and if not testing again for HIV 10 cp/100 μl. If HIV 100 and 10 c are not present, the system tests for HIV 1000 cp/100 μl. Two different models are used to screen for whole blood and HIV 1000 cp/100 μl, and thereafter S. aureus concentration can be detected. The system of the present invention in this example was able to achieve 100% whole blood differentiation, 100% HIV sample differentiation with specificity of 95%, 90% S. aureus differentiation, and the ability to discriminate quantitatively demonstrated.

### EXAMPLE 2

### HSV1 and HSV2 in Cerebral Spinal Fluid

In this example, models for sequentially testing for HSV1 and HSV2 in CSF were developed for deployment on LIBS instrumentation (consistent with the embodiment shown in FIG. 6), and were able to achieve 98% HIV differentiation with 100% specificity, and 85% HSV type differentiation.

Based upon this and other testing of the methods of the present invention, your inventors were able to demonstrate rapid, real-time clinical diagnostic analysis with minimal sample preparation, at clinically relevant levels, on samples excluded from algorithm development.

It is noted that the terms “substantially” and “about” may be utilized herein to represent the inherent degree of uncertainty that may be attributed to any quantitative comparison, value, measurement, or other representation. The terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A method for detecting parasites, viruses, bacteria or drugs in human blood, animal blood or cerebral spinal fluid, the method comprising the steps of:
a. Providing at least one sample of human blood, animal blood or cerebral spinal fluid;
b. Using a laser-induced breakdown spectroscopy instrument, (i) applying a laser to the sample; (ii) collecting light emitted from said sample; and (iii) generating spectrum from said collected light; and
c. Determining the presence or absence of parasites, viruses, bacteria or drugs, or combinations thereof, in the sample, by applying the generated spectra to one or more algorithmic models, wherein such models are developed from other spectra generated by a laser-induced breakdown spectroscopy instrument from samples of human blood, animal blood or cerebral spinal fluid with known presence of parasites, viruses, bacteria or drugs.

2. The method of claim 1, wherein the detection algorithm models are developed to differentiate S. aureus in blood.

3. The method of claim 1, wherein the detection algorithm models are developed to differentiate Leishmania donovani in blood.

4. The method of claim 3, wherein the detection algorithm models are developed to differentiate concentration of Leishmania donovani in blood.

5. The method of claim 1, wherein the detection algorithm models are developed to differentiate human immunodeficiency virus in blood.

6. The method of claim 1, wherein the detection algorithms are developed to differentiate herpes simplex virus in cerebral spinal fluid.

7. The method of claim 1, wherein the detection algorithms are further developed to differentiate parasite media or bacteria media.

8. The method of claim 1, wherein the spectra derive from multiple samples of blood or spinal fluid having known presence of the same parasite, virus, bacteria or drug.

9. A laser-induced breakdown spectroscopy instrument comprising a computer having algorithmic detection models for detecting parasites, viruses, bacteria or drugs in human blood, animal blood or cerebral spinal fluid, wherein said models are developed from and verified by spectra generated by a laser-induced breakdown spectroscopy instrument from samples of human blood, animal blood or cerebral spinal fluid with known presence of parasites, viruses, bacteria or drugs, said samples being positioned upon a substrate.

10. The device of claim 9, wherein the algorithmic detection models are specific to the substrate of the sample.

11. The device of claim 9, wherein the algorithmic detection models are specific to a condition of the sample at the time of the spectra are generated.

12. The device of claim 9, wherein the algorithmic detection models are designed to differentiate the drugs MLV vaccine and ATP vaccine.

13. A method for developing and using algorithmic detection models for detecting parasites, viruses, bacteria or drugs in human blood, animal blood or cerebral spinal fluid, the method comprising the steps of:
a. providing a first sample of human blood, animal blood or cerebral spinal fluid, said sample comprising one or more parasites, viruses, bacteria or drugs;
b. using a laser-induced breakdown spectroscopy instrument, (i) applying a laser to the sample; (ii) collecting light emitted from said sample; and (iii) generating spectrum from said collected light;
c. grouping some spectra so generated as classification spectra, and some spectra so generated as verification spectra;
d. developing one or more algorithmic models from said classification spectra;
e. verifying said one or more algorithmic models with said verification spectra;
f. providing a second sample of different human blood, animal blood or cerebral spinal fluid;
g. determining the presence or absence of parasites, viruses, bacteria or drugs, or combinations thereof, in the second sample, by applying the generated spectra to said one or more algorithmic models.

14. The method of claim 13, wherein the step of developing one or more algorithmic models from said classification spectra includes analyzing the spectra using elemental, molecular and background emissions over a wide spectral range of 200-1000 nm

15. The method of claim 13, wherein the step of developing one or more algorithmic models from said classification spectra includes analyzing the spectra using elemental, molecular and background emissions over selected wavelength ranges.

16. The method of claim 13, wherein the step of developing one or more algorithmic models from said classification spectra includes applying mathematical analysis such as chemometric analysis to at least a portion of the spectrum.

17. The method of claim 13, wherein the step of developing one or more algorithmic models from said classification spectra may include mathematical pretreatment of the spectra prior to creating differentiation models such as applying spectral normalization to the highest intensity feature in the spectrum or spectral normalization to the total area of the spectrum or other.

18. The method of claim 13, wherein the algorithm models are developed to differentiate concentration of Leishmania donovani in blood.

19. The method of claim 1, wherein the algorithm models are developed to differentiate herpes simplex virus in cerebral spinal fluid.
